(19) United States
(12) Patent Application Publication
(10) Pub. No.: US 2002/0132271 A1
(43) Pub. Date: Sep. 19, 2002

(54) Reagents, method and kit for detecting phosphinothricin-N-acetyltransferase protein

(76) Inventors: Dale V. Onisk, Bear, DE (US); James W. Stave, Elkton, MD (US); Alan B. McQuillin, Newark, DE (US)

Correspondence Address: John S. Pratt, KILPATRICK STOCKTON LLP, Suite 2800, 1100 Peachtree Street, Atlanta, GA, 30309-4530, US

(21) Appl. No.: 09/966,335

(22) Filed: Sep. 28, 2001

Related U.S. Application Data
(60) Provisional application No. 60/236,609, filed on Sep. 29, 2000.

(51) Int. Cl. G01N 33/573; G01N 33/53

(52) U.S. Cl. 435/7.4; 435/7.32; 435/7.8

(57) ABSTRACT

A method, kit and reagents for the detection of (phosphinothricin-N-acetyltransferase) PAT proteins in a sample, particularly a genetically modified agricultural crop sample. The proteins to be detected are one or more PAT enzyme proteins from various species of Streptomyces, including S. hygroscopicus and S. viridochromogenes. In particular, the PAT proteins are detected in genetically modified plants containing a gene, such as the pat or bar gene, that renders the plant resistant to the herbicide glufosinate. The reagents are antigenic peptides having common epitope of the PAT proteins from two or more Streptomyces species and antibodies immunoreactive with these proteins, particularly those expressed by the bar and pat genes. The peptides are isolated or synthesized and administered to animals to produce anti-PAT monoclonal and polyclonal antibodies having high sensitivity for PAT proteins. The antibodies are useful in immunoassay methods for the detection of genetically modified organisms engineered to include a PAT gene.

### CROSS REFERENCE TO RELATED APPLICATION

This application claims priority to U.S. Provisional Patent Application No. 60/236,609, filed Sep. 29, 2000.

### FIELD OF THE INVENTION

This relates to the field of immunology and more specifically relates to an immunoassay method, kit and reagents, for the detection of phosphinothricin-N-acetyltransferase protein.

### BACKGROUND OF THE INVENTION

Modem biotechnology techniques are being used to genetically modify many different species of plants, including large commercial commodities such as corn, cotton, soybean, wheat, and rice. The modified plants contain novel segments of DNA that result in the production of new proteins that impart novel characteristics to the crop. The novel DNA and proteins can be found to varying degrees in many parts of the modified plants, including leaves, seed and grain, and the processed fractions and final foods prepared from them. Plants that have been engineered in this fashion have been referred to as genetically modified organisms (GMO).

Examples of transgenic plants are insect and herbicide tolerant corn, cotton and soybeans. A number of different transgenic corn events have been produced that are resistant to specific herbicides. One such corn event sold by Aventis CropScience (Research Triangle Park, N.C.), referred to as event T25, is resistant to a non-selective herbicide (glufosinate) sold under the trade name Liberty®. Hybrid varieties of T25 corn resistant to Liberty herbicide are sold under the trademark LibertyLink®. Glufosinate-tolerant crops offer the advantage that farmers can spray their fields with glufosinate, killing the weeds and leaving the crop intact.

Traits such as insect and herbicide resistance have led to rapid acceptance of transgenic crops by farmers, especially in the United States. For example, approximately 30% of the corn planted in the United States in 1999 was transgenic. While farmers have accepted transgenic crops, food consumers do not perceive a direct benefit from genetically modified agricultural products. In fact, there is vigorous debate in the world community over the acceptance and use of genetically modified crops encompassing a very wide range of topics including international trade, environmental effects, safety, biodiversity, religious and ethical considerations, and the patenting of living organisms.

In the face of such controversy, countries have established laws that mandate the labeling of food as to its GMO content. Determining whether a plant has been genetically modified or whether grain or processed foods contain GMO requires test methods that can detect and quantitate either the novel DNA or protein. Immunoassays are used routinely in human diagnostics and clinical chemistry to detect and quantitate specific proteins in complex sample matrices, and immunoassays have been developed to detect transgenic proteins in genetically modified crops (Bauer-Weston et al., Plant Molecular Biology Reporter, 14(2), pp. 134-142,1996; Fuchs et al., in ANALYTICAL CHEMISTRY OF BACILLUS THURINGIENSIS, pp. 105-113, 1990; Stave, Food Control 10, pp. 367-374, 1999).

Resistance to glufosinate is accomplished by incorporating a gene into the DNA of the plant that encodes a particular protein enzyme. When produced within the cells of the plant, the enzyme modifies the herbicide rendering it non-toxic to the host. The enzyme is referred to as phosphinothricin-N-acetyltransferase, or PAT, and two different genes coding for this enzyme have been isolated from different species of Streptomyces. The gene isolated from S. hygroscopicus is referred to as the bar gene and the gene isolated from S. viridochromogenes is known as the pat gene. The PAT proteins encoded by the pat and bar genes share approximately 85% amino acid sequence homology (Wohlleben et al., Gene 70, pp. 25-37, 1988).

Not only is resistance to glufosinate useful to farmers, but resistance to glufosinate is used extensively by researchers developing new transgenic plants as a way to select cells that have successfully incorporated novel DNA. To do this, scientists attach a glufosinate-resistance gene to a second gene coding for a desired characteristic and insert the DNA containing both genes into plant cells grown in tissue culture. The media used to grow these cells contains glufosinate. If a cell successfully incorporates the novel DNA, then it is capable of growing in the glufosinate-containing media. Cells that have not incorporated the DNA die. Using glufosinate as a selectable marker, researchers have made many transgenic plants. Thus, while resistance to glufosinate was not the intended agronomic trait, many transgenic plants contain the PAT protein because it was used in the development process to select for successfully transformed cells.

It is important to recognize that transgenic plant developers have used both the pat and bar genes to confer glufosinate resistance. Of the five major corn events in commercial production today, three contain the bar gene (MON810,CBH351, DBT418), and two contain the pat gene (BT11, T25). Other examples of PAT-containing crops include canola (pat and bar varieties), rice (bar), soybean (pat and bar), and many others. From the large numbers of crops and varieties that express the PAT protein it is clear that a single test or assay is needed that can detect PAT proteins expressed from both the pat gene and the bar gene. A currently available commercial immunoassay (PAT ELISA, Steffens Biotechnische Analysen GmbH, Ebringen, Germany) employing antibodies made to PAT protein expressed from the pat gene is only useful for detecting PAT from the pat gene, not PAT expressed from the bar gene as shown in FIG. 1 and FIG. 2. This lack of crossreactivity renders the available test useless for the detection of PAT protein from both the pat and bar genes. Therefore, antibodies, reagents, and high sensitivity tests capable of detecting low concentrations of transgenic PAT protein expressed from both the pat and bar genes are needed.

### SUMMARY OF THE INVENTION

A method, kit and reagents for detecting and measuring phosphinothricin-N-acetyltransferase (PAT) protein in a sample are provided. The proteins to be detected are one or more PAT enzyme proteins from various species of Streptomyces, including S. hygroscopicus and S. viridochromogenes. In particular, the PAT proteins are detected in genetically modified plants containing a gene, such as the pat or bar gene, that renders the plant resistant to the herbicide glufosinate.

The reagents include antigenic peptides and antibodies. The antigenic peptides are immunoreactive with the monoclonal antibodies 98AD8, 98AY4 and 98BA12, described in more detail below. The antigenic peptides have common epitopes shared by PAT proteins encoded by genes from different species of Streptomyces. The peptides are isolated or synthesized and administered to animals to produce anti-PAT monoclonal and polyclonal antibodies.

The antibodies have high sensitivity and crossreactivity for PAT proteins from various species and are therefore useful in immunoassay methods for the detection of genetically modified organisms, particularly plants, which have been engineered to include a PAT gene. The preferred antibodies are the monoclonal antibodies 98AD8, 98AY4 and 98BA12.

The methods are immunoassays employing antibodies described herein and are capable of detecting low concentrations of PAT protein in genetically enhanced crop samples. The antibodies are immunoreactive with epitopes or common epitopes on PAT expressed by both the pat and bar genes and react minimally with other proteins that may be present in the sample, thus providing for an accurate determination of the presence of a genetically modified organism in a sample, such as a grain sample.

The epitopes, antibodies, or both, are collectively assembled in a kit with conventional immunoassay reagents for detection of PAT protein. The kit may optionally contain both monoclonal and polyclonal antibodies and a standard for the determination of the presence of PAT protein in a sample.

It is therefore an object of the present invention to provide reagents, immunoassay methods, and kits for the detection of PAT protein in a sample, particularly a genetically modified agricultural sample from a plant transfected with a gene expressing PAT derived from any Streptomyces species.

It is a further object of the present invention to provide a highly sensitive immunoassay for PAT protein.

It is a further object of the present invention to provide an antigenic peptide for the production of antibodies highly specific for PAT protein.

It is a further object of the present invention to provide high affinity antibodies for the PAT proteins expressed from genes from various strains of Streptomyces that exhibit minimal crossreactivity with other proteins.

These and other objects of the present invention will become apparent after reading the following detailed description of the disclosed embodiments and the appended claims.

### BRIEF DESCRIPTION OF THE DRAWINGS

FIG. 1 is a graph showing the results (absorbance versus concentration) of a commercially available assay (PAT-ELISA, Steffens Biotechnische Analysen GmbH, Ebringen, Germany) for the detection of various concentrations of PAT protein expressed from the pat and bar genes.

FIG. 2 is a graph showing the results (absorbance versus % GMO) of the commercially available assay of FIG. 1 for the detection of various concentrations of PAT protein in four genetically modified corn seed extracts, T25 (Pioneer, Des Moines, Iowa) GA21 (Monsanto, St. Louis, Mo.), 176 (Hoffman Seeds Inc., Lancaster, Pa.) and Mon810 (Pioneer, Des Moines, Iowa).

FIG. 3 is a graph showing the results of an epitope mapping experiment with the monoclonal antibodies 98AD8, 98BA12 and 98AY4.

FIG. 4 is a graph of absorbance versus percent GMO showing reactivities of various GMO Corn Seed Extracts in an ELISA.

FIG. 5A is a graph of absorbance versus monoclonal antibody concentration showing direct bind of various monoclonal antibodies with PAT expressed from the pat gene. FIGS. 5B is a graph of absorbance versus monoclonal antibody concentration showing direct bind of various monoclonal antibodies with PAT expressed from the bar gene.

FIG. 6 is a graph of percent inhibition versus inhibitor concentration showing direct bind with PAT inhibition wherein PAT is expressed from the pat gene.

FIG. 7 is a graph of percent inhibition versus inhibitor concentration showing direct bind with PAT inhibition wherein PAT is expressed from the bar gene.

FIG. 8A is a graph of absorbance versus PAT protein concentration (expressed from the pat gene) showing the crossreactivity of various monoclonal antibodies. FIG. 8B is a graph of absorbance versus PAT protein concentration (expressed from the bar gene) showing the crossreactivity of various monoclonal antibodies.

FIG. 9 is a graph of absorbance versus dilution factor of extract showing the reactivity of various monoclonal antibody-biotin conjugates with various monoclonal antibodies.

FIG. 10 is scanned reproduction of Western blots showing reactivity of three monoclonal antibodies and a control antibody with PAT protein expressed from the bar gene and the pat gene, and molecular weight markers.

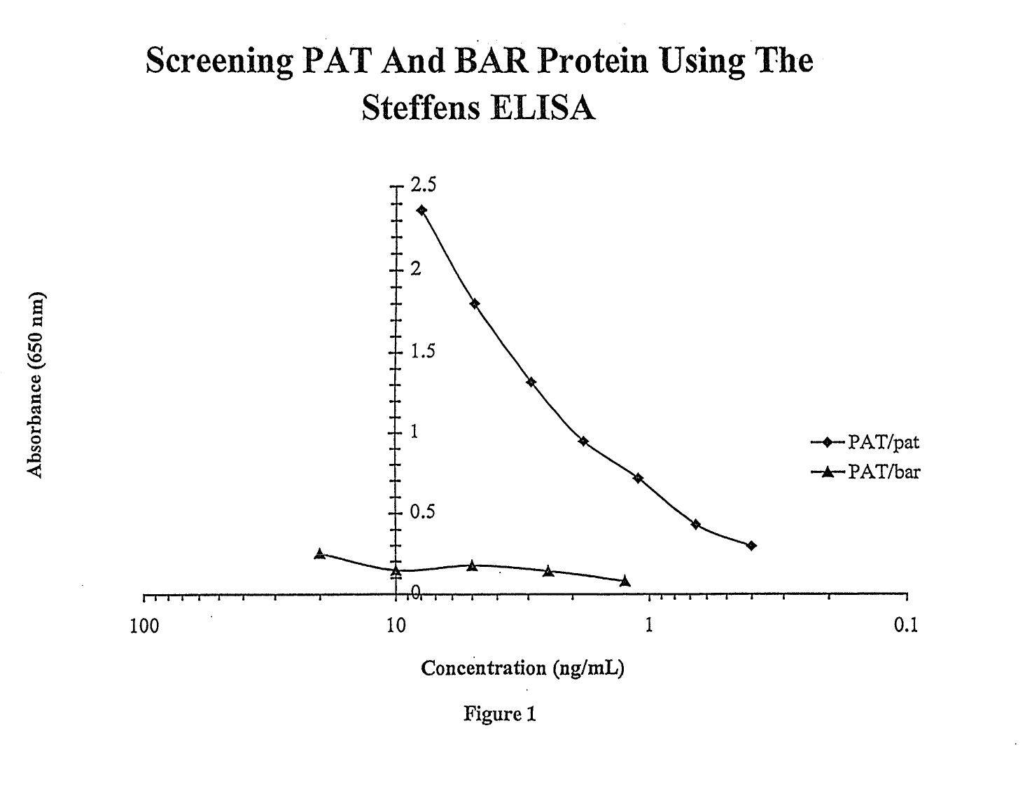

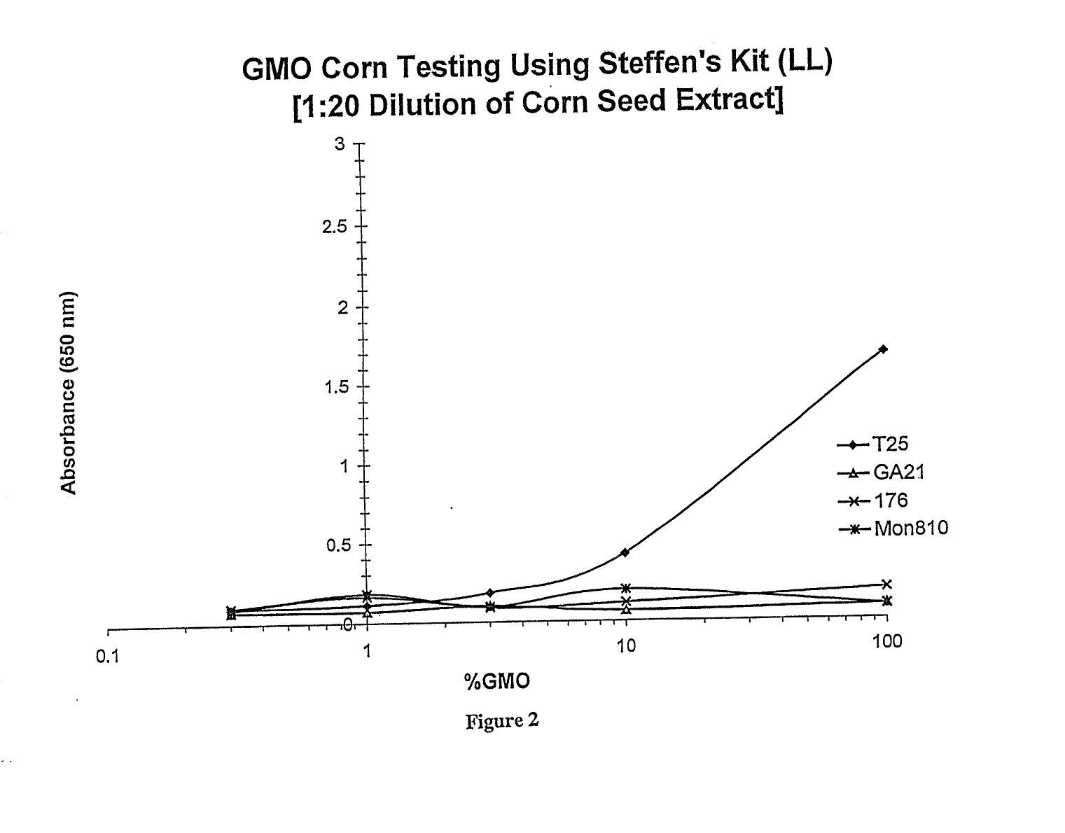

### DETAILED DESCRIPTION OF THE DISCLOSED EMBODIMENTS

A method, kit, and reagents for the detection of pbosphinothricin-N-acetyltransferase (PAT) proteins in a sample are described herein. The PAT protein confers resistance to the herbicide glufosinate.

It is important when making immunoassays to detect PAT protein in transgenic plants and the products produced from them (including food fractions), that a test have the capacity to detect the PAT protein from both the pat and bar genes because transgenic plants have been made with both genes. Thus, crossreactive antibodies are very important for development of successful commercial products.

The reagents are antigenic peptides of PAT proteins sharing common epitopes and anti-PAT antibodies that are crossreactive with PAT proteins expressed from different genes. The method is an immunoassay for the sensitive, specific detection of PAT protein, specifically for the detection of PAT protein expressed from genetically engineered plants, such as agricultural products. The kit contains the anti-PAT antibodies described herein and other reagents, particularly those used in a strip test format, for use in the immunoassay described in more detail below.

### ANTIGENIC PEPTIDES

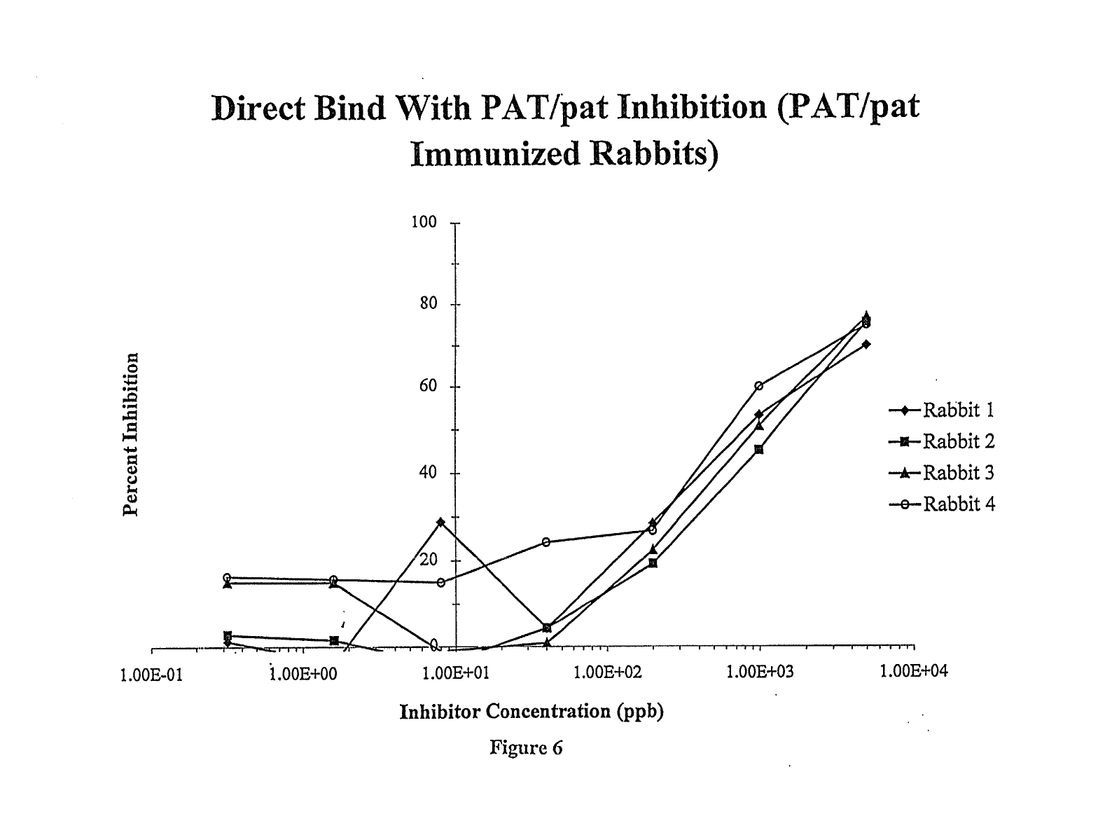

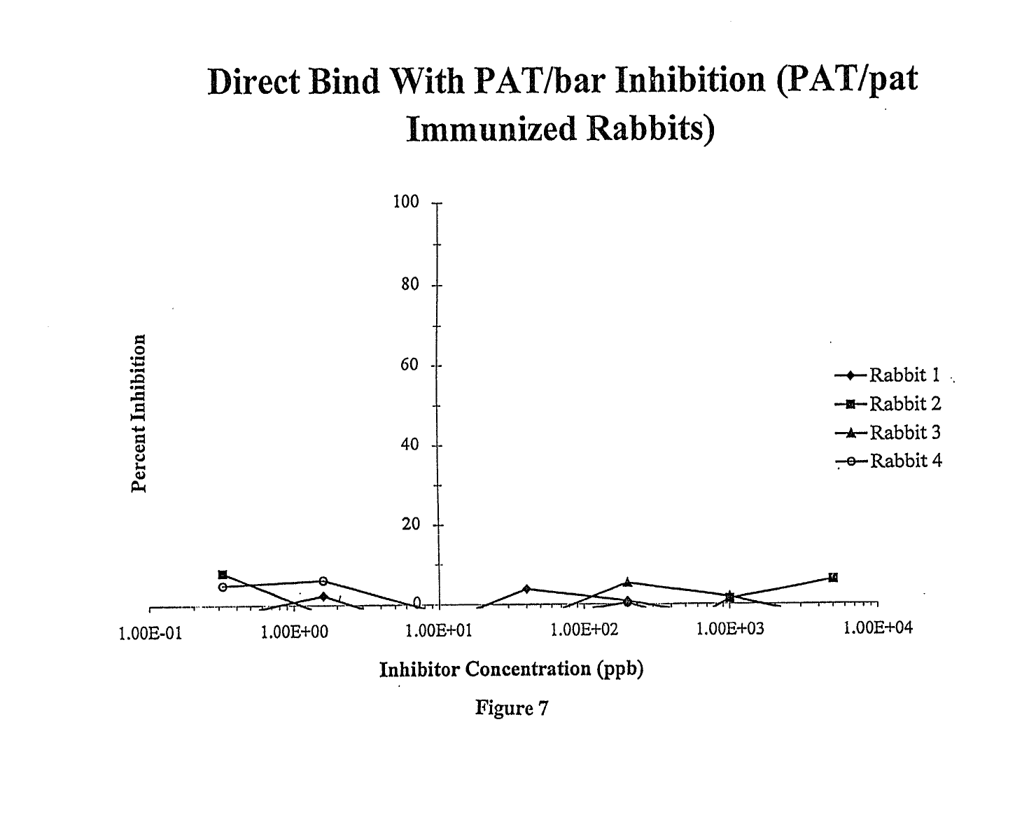

The antigenic peptides are PAT protein surface peptides that share epitopes across various species expressing the protein, preferably protein expressed from various Streptomyces strains, most preferably from both S. hygroscopicus and S. viridochromogenes. The peptides are not immunodominant, as evidenced by the lack of crossreactivity and sensitivity of polyclonal antibodies raised against the whole protein as shown in FIG. 6 and FIG. 7.

The peptides are highly useful as diagnostic markers for the detection and quantification of the PAT protein. The peptides are also useful for producing antibodies, tests and kits having the superior sensitivity required of successful commercial products.

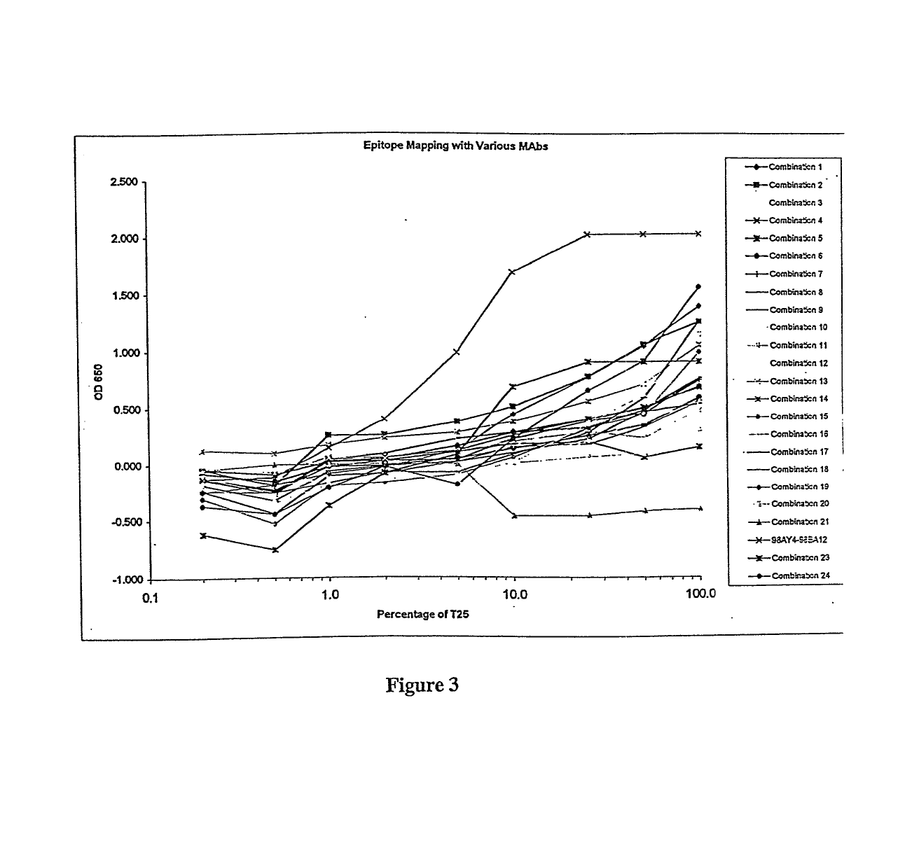

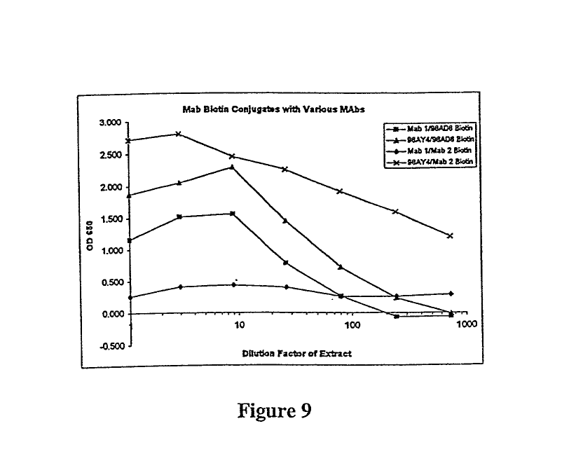

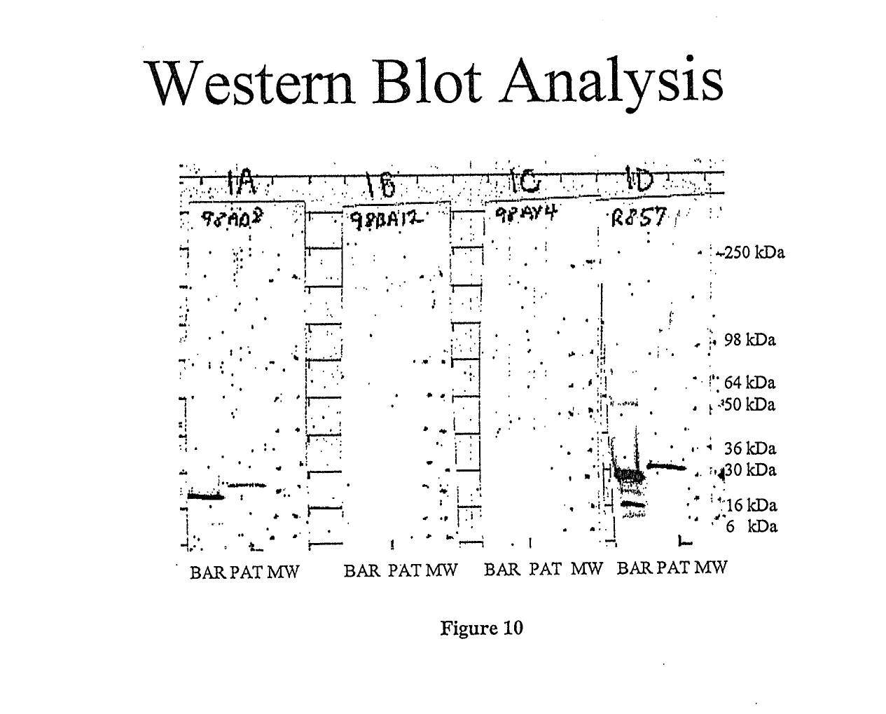

The peptides are both linearly and conformationally antigenic as determined by the presence and lack of Western blot reactivity with the monoclonal antibodies described herein. For example, a monoclonal antibody (98AD8, described below) binds PAT in Western blot and therefore recognizes a linear epitope. In contrast, a monoclonal antibody (98BA12, described below) fails to bind to the PAT protein in Western blot and therefore recognizes a conformationally-determined epitope. These results are shown in FIG. 10. The results of epitope mapping experiments with these two and a third antibody (98AY4, described below) demonstrate that all three antibodies recognize different epitopes as shown in FIG. 3 and FIG. 9. The existence of three spatially distinct, crossreactive epitopes on the surface of a small molecular weight protein (approximately 24,000 Da) is highly surprising.

The peptides are either isolated from cell cultures in which the PAT-encoding genes are expressed using conventional techniques known to those skilled in the art such as affinity column purification or the amino acid sequences of the peptides are generated and the peptides synthesized in accordance with methods known to those in the art. The proteins to be detected are the PAT proteins from various species of Streptomyces, including S. hygroscopicus and S. viridochromogenes. including the pat and bar genes and antibodies immunoreactive with those peptides or epitopes.

### Antibodies

During the development of an assay to detect the PAT protein in genetically enhanced crops (such as corn), great difficulty was encountered in finding a single polyclonal or monoclonal antibody having sensitive immunoreactivity to PAT protein expressed from various genes. For example, polyclonal antibodies raised to PAT protein from either the pat or bar gene showed minimal crossreactivity to protein from the heterologous gene as shown in FIG. 6 and FIG. 7. Immunoassays developed using these antibodies had relatively poor sensitivity. Lack of crossreactivity was surprising because the proteins from these two genes share 85% amino acid sequence homology. Based on these results, it was assumed that monoclonal antibodies made to the PAT protein would have even less crossreactivity.

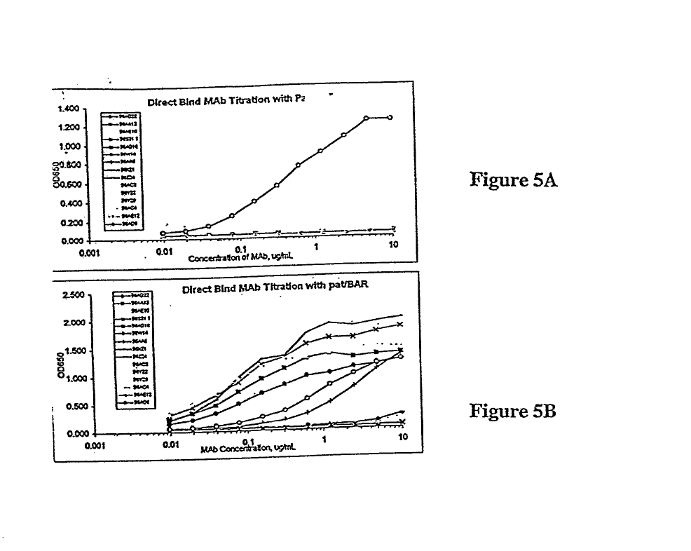

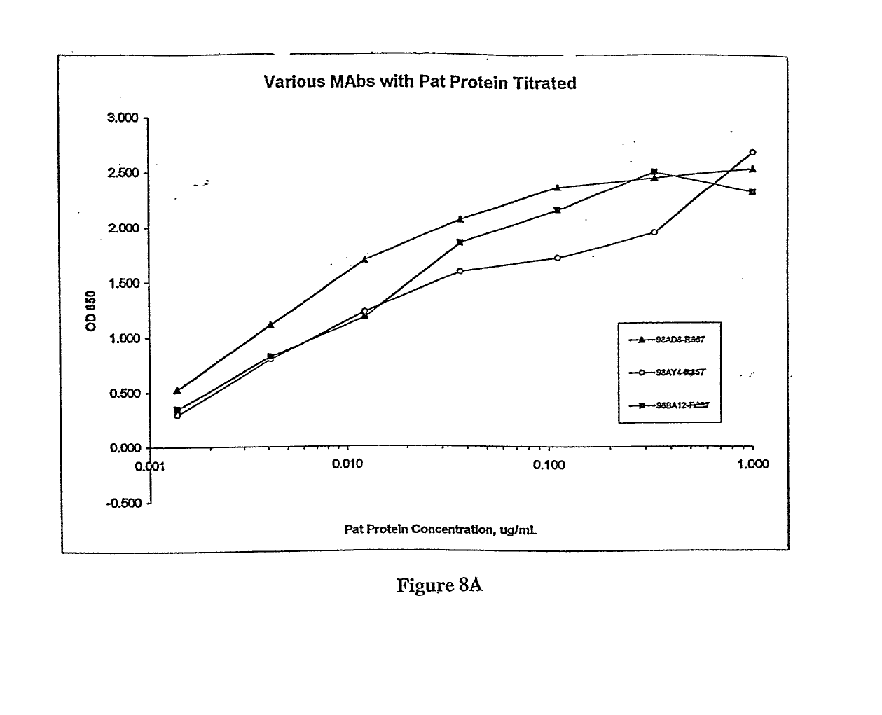

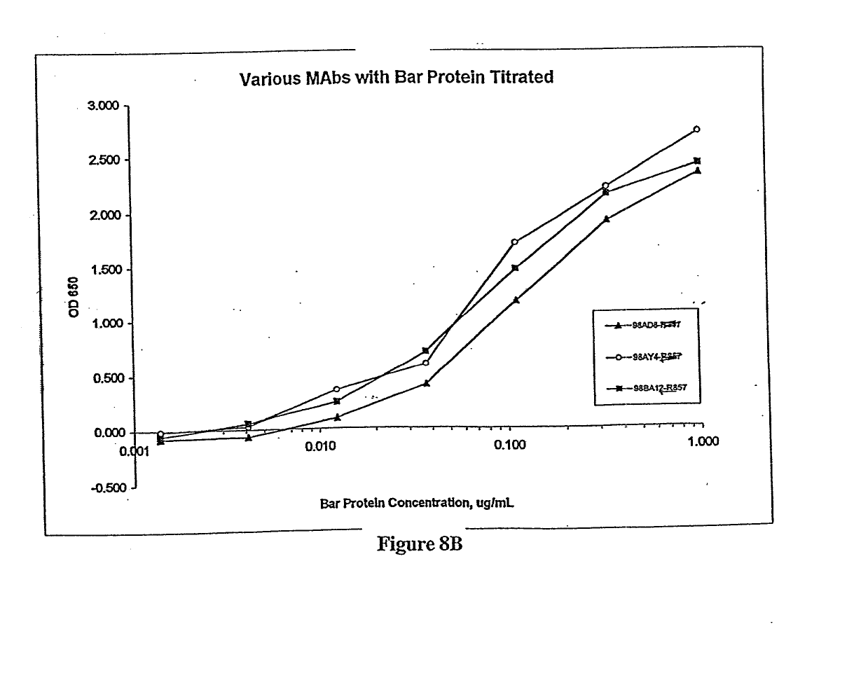

However, the antibodies provided herein are crossreactive with PAT protein expressed from genes derived from various organisms, preferably two or more Streptomyces species, as shown in FIG. 5. Most preferably, the antibodies crossreact with PAT proteins expressed from both the S. hygroscopicus and the S. viridochromogenes genes, namely the pat gene and the bar gene, as shown in FIG. 8.

The preferred antibodies are highly sensitive for the detection of PAT proteins, particularly transgenic PAT proteins at relevant concentrations in bulk samples of commodity grain in the distribution channel. Preferably, the antibodies detect PAT protein expressed from both the pat gene and the bar gene at a high sensitivity of 1 ng/mL. High sensitivity antibodies are required for detection of low concentrations of PAT proteins in genetically engineered crop tissues, such as, but not limited to, leaf, stem, seed, stalk, root, and the like, or products derived from such crops, such as food fractions.

Antigenic peptides having the characteristics set forth above are useful for the production of both monoclonal or polyclonal antibodies reactive with the PAT protein. The preferred antibody is a monoclonal antibody, due to its higher specificity for analyte.

Monoclonal antibodies are generated by methods well known to those skilled in the art. The preferred method is a modified version of the method of Kearney, et al., J Immunol. 123:1548-1558 (1979), which is incorporated by reference herein. Briefly, animals such as mice or rabbits are inoculated with the immuogen in adjuvant, and spleen cells are harvested and mixed with a myeloma cell line, such as P3X63Ag8,653. The cells are induced to fuse by the addition of polyethylene glycol. Hybridomas are chemically selected by plating the cells in a selection medium containing hypoxanthine, aminopterin and thymidine (HAT). Hybridomas are subsequently screened for the ability to produce anti-PAT monoclonal antibodies. Hybridomas producing antibodies are cloned, expanded and stored frozen for future production.

The antibody may be labeled directly with a detectable label for identification and quantitation of PAT protein. Labels for use in immunoassays are generally known to those skilled in the art and include enzymes, radioisotopes, and fluorescent, luminescent and chromogenic substances including colored particles such as colloidal gold and latex beads.

Alternatively, the antibody may be labeled indirectly by reaction with labeled substances that have an affinity for immunoglobulin, such as protein A or G or second antibodies. The antibody may be conjugated with a second substance and detected with a labeled third substance having an affinity for the second substance conjugated to the antibody. For example, the antibody may be conjugated to biotin and the antibody-biotin conjugate detected using labeled avidin or streptavidin. Similarly, the antibody may be conjugated to a hapten and the antibody-hapten conjugate detected using labeled anti-hapten antibody. These and other methods of labeling antibodies and assay conjugates are well known to those skilled in the art.

Preferably, the antibodies are the monoclonal antibodies 98AD8, 98AY4 and 98BA12, produced by hybridomas deposited with the American Type Culture Collection, Rockville, Md. on or before Apr. 10, 2001. The hybridoma producing monoclonal antibody 98AD8 is deposited as ATCC Accession No. PTA-3266. The hybridoma producing monoclonal antibody 98AY4 is deposited as ATCC Accession No. PTA-3267. The hybridoma producing monoclonal antibody 98BA12 is deposited as ATCC Accession No. PTA-3265. Anti-PAT monoclonal and polyclonal antibodies having similar or superior sensitivity for PAT proteins are produced by inununization of an animal with the PAT peptides described above, isolation of antibodies that react with the peptides, and the collection and purification of the antibodies from a biological fluid such as blood in accordance with methods well known to those skilled in the art.

The antibodies are collectively assembled in a kit with conventional immunoassay reagents for detection of PAT protein using the immunoassay described below. The kit may optionally contain both monoclonal and polyclonal antibodies and a standard for determining the presence of PAT in a sample. The kit containing these reagents provides for simple, rapid, on site detection of PAT protein.

### IMMUNOASSAY

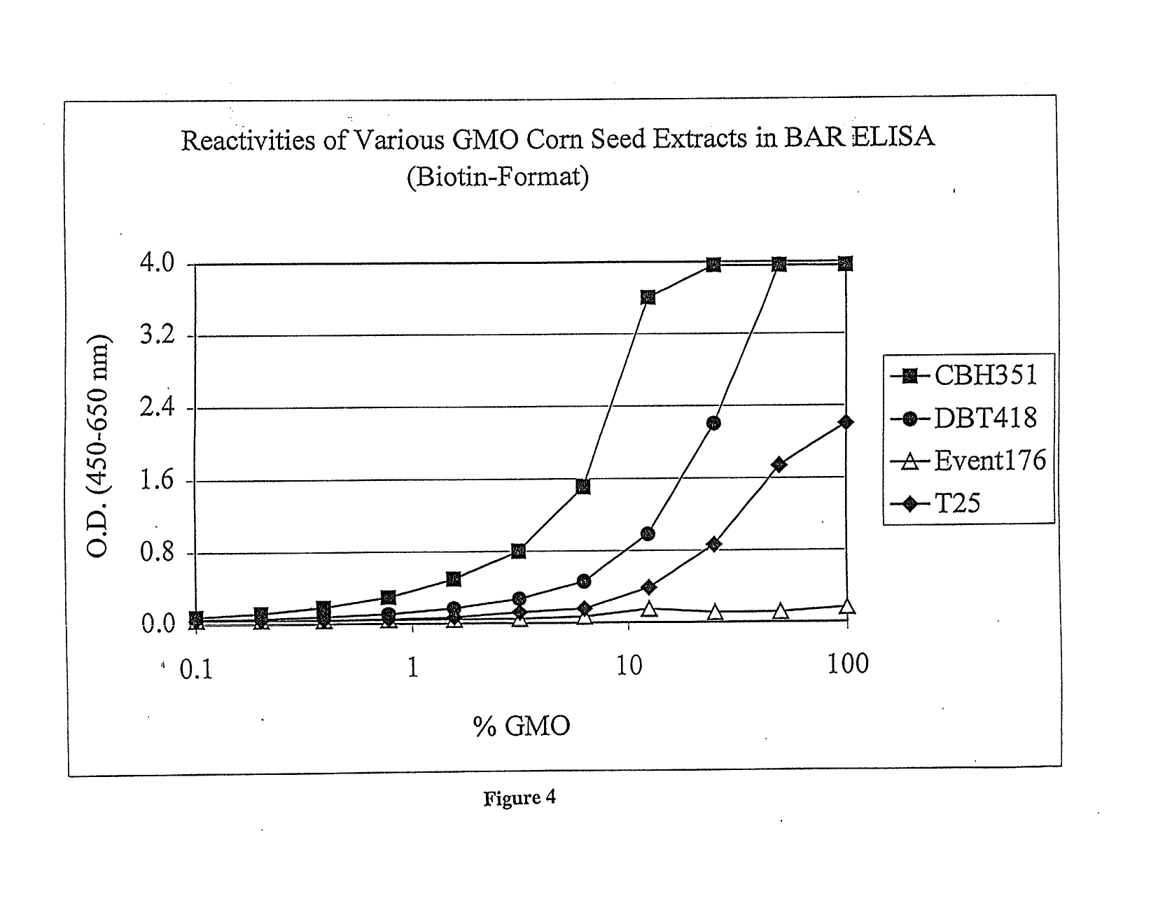

A highly sensitive immunoassay employing the antibodies described above is provided. The assay is useful for the detection of genetically modified organisms that have been engineered to include a PAT gene. The immunoassay is capable of detecting low concentrations of PAT protein in genetically enhanced crop samples. As described above, the antibodies used in the immunoassay are immunoreactive with epitopes or a common epitope on the PAT protein expressed by two or more Streptomyces species genes, particularly both the pat and bar genes, and react minimally with other proteins that may be present in the sample, thus providing for an accurate determination of the presence of a genetically modified organism in a sample, such as a grain sample. For example, the preferred assay can detect a transgenic product, such as T25 grain (transgenic corn resistant to glufosinate) in an amount less than or equal to 1% GMO in composite corn samples as, shown in FIG. 4.

The immunoassay is useful for detecting the presence or amount of PAT in a variety of samples, particularly agricultural samples such as plant material, particularly agricultural samples. The sample may be obtained from any source in which the PAT proteins are accessible to the antibody. For example, the sample may be any plant tissue or extract including root, stem, stalk, leaf, or seed or products derived from such crops, such as food fractions.

One or more of the antibodies described above may be employed in any heterogeneous or homogeneous, sandwich or competitive immunoassay for the detection of PAT protein. Either the antibody is labeled with a detectable label or coupled to a solid phase. Methods for coupling antibodies to solid phases are well known to those skilled in the art. In accordance with the immunoassay method, the sample containing the analyte is reacted with the antibody for a sufficient amount of time under conditions that promote the binding of antibody to PAT protein in the sample. It will be understood by those skilled in the art that the immunoassay reagents and sample may be reacted in different combinations and orders. A physical means is employed to separate reagents bound to the solid phase from unbound reagents such as filtration of particles, decantation of reaction solutions from coated tubes or wells, magnetic separation, capillary action, and other means known to those skilled in the art. It will also be understood that a separate washing of the solid phase may be included in the method.

The concentration of PAT protein in the sample is determined either by comparing the intensity of the color produced by the sample to a color card or by using a reflectometer.

The resulting reaction mixture, or combination of antibody and sample, is prepared in a solution that optimizes antibody-analyte binding kinetics. An appropriate solution is an aqueous solution or buffer. The solution is preferably provided under conditions that will promote specific binding, minimize nonspecific binding, solubilize analyte, stabilize and preserve reagent reactivity, and may contain buffers, detergents, solvents, salts, chelators, proteins, polymers, carbohydrates, sugars, and other substances known to those skilled in the art.

The reaction mixture solution is reacted for a sufficient amount of time to allow the antibody to react and bind to the analyte to form an antibody-analyte complex. The shortest amount of reaction time that results in binding is desired to minimize the time required to complete the assay. An appropriate reaction time period for an immunochromatographic strip test is less than or equal to 20 minutes or between approximately one minute and 20 minutes. A reaction time of less than five minutes is preferred. Most preferably, the reaction time is less than three minutes. By optimizing the reagents, binding may be substantially completed as the reagents are combined.

The reaction is performed at any temperature at which the reagents do not degrade or become inactivated. A temperature between approximately 4° C. and 37° C. is preferred. The most preferred reaction temperature is ambient or room temperature (approximately 25° C.).

A chromatogenic test strip is ideally suited for this immunoassay. Test strips are comprised of multiple porous components, membranes and filters, through which liquid sample is drawn by capillary action. Analyte in the sample reacts with the test reagents contained within the test strip as it traverses the length of the strip. To detect protein in grain or seed, the grain is ground into a powder and the protein extracted from the powder with a liquid that is then separated from the solid material and assayed using the test. The liquid is applied to the chromatographic strip, and the analyte migrates toward the distal end of the strip. As it migrates down the strip, the analyte reacts with reagents applied to or immobilized on the strip causing a detectable signal product. Detection of the signal indicates the presence of the analyte in the sample.

### Immunoassay Kit

An immunoassay kit for the detection of PAT protein in a sample contains one or more of the antibodies described above.

The kit may additionally contain equipment for obtaining the sample, a vessel for containing the reagents, a timing means, a buffer for diluting the sample, and a colorimeter, reflectometer, or standard against which a color change may be measured. The kit may include the reagents in the form of a chromatographic test strip as described above.

In a preferred embodiment, the reagents, including the antibody are dry. Addition of aqueous sample to the vial or strip results in solubilization of the dry reagent, causing it to react.

The reagents, immunoassay method, and kit described above will be further understood with reference to the following non-limiting examples.

### EXAMPLE 1

### PAT Epitope Mapping

An experiment was performed to map the PAT epitope.

1. Coat two NUNC Maxisorp plates at 5 μg/mL (100 μL/well) PAb R350-351 in 0.1 M carbonate. Incubate one hour at 37° C.

2. Dump plates and pat dry.

3. Block with PCT (PBS, 1% casein, pH 7.5).

4. Incubate 30 minutes or more at 37° C. Wash three times with PT (PBS, 0.05% Tween20, pH 7.5)

5. Add 100 μL/well of BT11 corn seed extract at 1:100 dilution in PCT.

6. Incubate one hour at 37° C. Wash. 7. Titrate monoclonal antibodies down plates at μL/well (starting concentration 20 μg/mL) and 1:3 down in PCT.

8. Incubate one hour at 37° C. Wash.

9. Add 0.2 μg/mL dilution of monoclonal antibody-Biotin Conjugate at 100 μg/well in PCT.

10. Incubate one hour at 37° C. Wash.

11. Add 1:2000 dilution of Streptavidin-horse radish peroxidase conjugate in PCT.

12. Incubate one hour at 37° C. Wash.

13. Add 100 μg/well of tetramethylbenzidine.

The results are shown in FIG. 3.

### EXAMPLE 2

### Analysis of PAT Epitopes by Western Blot

Antigenic peptides, or epitopes, of PAT proteins immunoreactive with the monoclonal antibodies 98AD8, 98BA12 and 98AY4 were analyzed by Western Blot to determine whether the epitopes were linear or conformationally antigenic.

### SDS-PAGE of pat and bar expressed PAT proteins

The following samples were prepared at the listed concentrations in Laemmli sample buffer with 2-ME:

| PAT/pat antigen (frozen) | PAT/bar (Gene B Protein) |
|---|---|
| 1.5 mg/ml 9/1/2000 | 11.3 mg/ml 9/14/2000 |
| Sample: 25 ng/15 ul | Sample: 25 ng/15 ul |

The samples were boiled for five minutes and dilutions were done in Laemmli sample buffer with 2-ME.

One 4-15% Tris-HCl gel (12 wells, 20 μl capacity, Cat. # 161-1176, Exp. 11/29/2000) was run at 100 V for about 1 hour.

| Lane | Sample | volume/well (μl) |
|---|---|---|
| 1 | See Blue | 5 |
| 2 | PAT/pat | 15 |
| 3 | PAT/bar | 15 |
| 4 | See Blue | 5 |
| 5 | PAT/pat | 15 |
| 6 | PAT/bar | 15 |
| 7 | See Blue | 5 |
| 8 | PAT/pat | 15 |
| 9 | PAT/bar | 15 |
| 10 | See Blue | 5 |
| 11 | PAT/pat | 15 |
| 12 | PAT/bar | 15 |

Gel was rinsed with distilled water for about 30 minutes.

Gel was stained for about one hour with one pumpful of Gelcode Blue Stain solution, then destained with distilled water overnight.

### Immunoblots

Gel was soaked in transfer buffer for about 60 minutes.

Transfer to nitrocellulose was at 100 V for one hour.

Ponceau S solution (5 ml) was added to the blot, and prominent bands were marked for reference.

Membrane was blocked with 5% NFDM in TBS, pH 8.0 overnight at 4° C.

Membrane was cut apart into four sections.

98AD8 was added to blot #1A at 10 μg/ml in 1% NFDM in TBS, pH 8.0 (15 ml).

98BA12 pool was added to blot #1B at 10 μg/ml in 1% NFDM in TBS, pH 8.0 (15 ml).

98AY4 was added to blot #1C at 10 μg/ml in 1% NFDM in TBS, pH 8.0 (15 ml).

857 pool was added to blot #1D at 10 μg/ml in 1% NFDM in TBS, pH 8.0 (15 ml).

Incubate for 1 hour at RT with shaking.

Wash for 30 minutes with TBS, 0.05% Tween 20.

To blot add 15 ml AP-Rabbit anti-mouse IgG (H+L) at 1:3000 in 1% NFDM in TBS, pH 8.0 to #1 A-C.

To blot add 15 ml of AP-Goat anti-rabbit IgG (H+L) at 1:3000 in 1% NFDM in TBS, pH 8.0 to #1D.

Incubate for 1 hour at RT with shaking.

Wash for 30 minutes with TBS, 0.05% Tween 20.

Add 10 ml BCIP/NBT substrate to each blot until bands develop.

Stop reaction by rinsing membranes with distilled water.

The results are shown in FIG. 10.

### EXAMPLE 3

### PAT Inhibition Assay

An Immulon 2 plate was coated overnight with PAT protein at 1.0 μg/mL in 0.1 M Carbonate buffer pH 9.0. 100 μl/well.

1. The plate was washed with PBS pH 7.2—0.5% Tween 20 (PT) using a plate washer.

2. The plate was blocked with PBS pH 7.2—1% Casein—0.5%

3. Tween 20 (PCT) was added, 130 μl/well, for 1 hour at room temperature (RT) with shaking (orbital shaker at 150 rpm).

4. The plate was washed with PBS pH 7.2—0.5% Tween 20 (PT) using a plate washer.

5. Titrate PAT/pat or PAT/bar antigen down the plates (50 μL/well, starting at 10 μmL) 1:5 down in PCT.

6. Add rabbit sera at 1:12,500 dilution in PCT to each well (50 μL/well).

7. The plate was incubated 1 hour at RT with shaking.

8. The plate was washed with PT as in Step 2.

9. The detecting conjugate, Goat anti-Rabbit IgG (H&L)-horseradish peroxidase (HRPase), was diluted 1:3000 in PCT and 100 μl/well was added. The plate was incubated for 1 hour at RT with shaking.

10. The plate was washed as in Step 2.

11. 100 μl/well of one component TMB substrate (KPL) was added. The plate was incubated ˜15 minutes at RT with shaking.

12. The plate was read at 650 nm using a plate washer. The results are shown in FIGS. 6 and 7.

### EXAMPLE 4

### Assay for PAT in Microtiter plate Format Using Monoclonal Antibodies

An immunoassay was performed for the detection of PAT as follows:

### Plate coating procedure

Monoclonal antibodies isolated from mice immunized with PAT protein expressed from the bar gene were prepared at 2.5 μg/ml in phosphate buffered saline (PBS) for coating. An aliquot of 100 μl per well was added to Nunc Maxisorp wells (C12), sealed with plate sealer, and incubated overnight at 4° C.

The following day, the contents of the wells were discarded and blocked with

1% bovine serum albumin (BSA) in PBS with 0.1% Tween 20.

### SAMPLES

Samples were prepared by grinding grain or seed in mortar with a pestle, then adding 10 ml of Traitcheck™ buffer (Strategic Diagnostics, Inc., Newark, Del.). The sample was spun in microfuge tubes to clear (15 K for 5 minutes). The procedure is as follows:

Wells were washed three times with plate washer.

100 μl of sample were added to wells and incubated 1 hour at 37° C.

Wells were washed six times with plate washer.

100 μl of monoclonal antibody in BSA blocking buffer was added.

Reactants were incubated 1 hour at 37° C. and washed six times with plate washer.

100 μl per well of horse radish peroxidase (HRP) Mouse anti-rabbit (Jackson) at 1/4000 in BSA blocking buffer was added.

Plates were washed six times with plate washer.

Tetramethylbenzidine (TMB, KPL) was added and plates read at 650 nm after 20 minutes.

### EXAMPLE 5

### Analysis of GMO Corn Using BAR ELISA

An enzyme linked immunoassay was used to analyze a corn sample for the presence of genetically modified organism (GMO) corn.

1. Create a desirable percentage of GMO to non-GMO using kernel to kernel ratios:

2. Add samples to Mason jars and grind using a Waring blender. A fine powder is obtained by further grinding with a coffee mill.

3. From each percentage to be tested, add 0.4 gram of the powder to a 2 mL microcentrifuge vial. Then transfer 1 mL of 10 mM PBS-0.05% Tween 20 buffer (PBST) (Ph7.2) to the vial and vortex vigorously for approximately 20 seconds.

4. Let vial incubate at room temperature for five minutes and centrifuge at 5,000 rpm for five minutes.

### Microtiter plate Preparation

1. Add 100 μL of 3 μg/mL of monoclonal antibody in 50 mM sodium carbonate coating buffer (pH 9.6) to each well of microtiter plate.

2. Incubate microtiter plate overnight at 4° C.

3. Pour out coating solution and block each well of microtiter plate with 200 μL of blocking solution [10 mM Tris buffer containing 0.02% (w/v) sodium caseinate, 5% (w/v) sucrose; pH 8.3].

4. Incubate microtiter plate at 37° C. for two hours.

5. Pour out blocking solution and blot remaining liquids from microtiter plate with dry paper towel.

6. Allow microtiter plate to stand in dry room overnight.

### Assay Procedure

1. Pipette 100 μL of supernatant from microcentrifuge vial and deliver to sample well of microtiter plate.

2. Incubate microplate at room temperature for 15 minutes.

3. Aspirate and wash microplate two times each way (with reverse direction).

4. Pipette 100 μL of monoclonal antibody-biotin conjugate (1:3200 dilution in PBST) to each sample well of microplate and allow incubation to proceed at room temperature for 15 minutes.

5. Aspirate and wash microplate two times each way (with reverse direction).

6. Add 100 μL of streptavidin HRP conjugate (1:64000 dilution in PBST) to sample well of microplate and incubate at room temperature for 15 minutes.

7. Aspirate and wash microplate two times each way (with reverse direction).

8. Add 100 μL of TMB substrate to each well of microplate and allow color reaction to proceed at room temperature for 20 minutes.

9. Stop the reaction with 100 μL of stop solution [0.5% (v/v) sulfuric acid].

10. Read the optical density (O.D.) of microplate at 450 nm with subtraction of 650

The results are shown in FIG. 4.

### EXAMPLE 6

### Analysis of GMO Corn Using Strip Test

An immunochromatographic strip test was used to analyze a corn sample for the presence of genetically modified organism (GMO) corn.

### Procedure

Extracts of corn were prepared by grinding 39 grams of corn to a fine powder. 10 grams of powder was added to a 50 ml centrifuge tube along with 40 ml of Traitcheck™ buffer (0.1% Tween, 0.1 M phosphate, pH 7.4, Strategic Diagnostics, Inc., Newark, Del.) and shaken for 15 minutes at room temperature. Large particulates were removed by centrifugation at 3000×g for 10 minutes and the supernatant removed for assay. Extracts were further diluted as indicated in Traitcheck™ buffer for assay.

### Assay

Three centimeter wide by 35 cm long nitrocellulose strips (Millipore SXHF) were sprayed with rabbit anti-PAT at 2 μg/cm at a distance of 1.25 mm from the bottom of the strip. Strips were mounted onto plastic backing with a wicking pad positioned on one edge and cut into 5.5 mm wide pieces.

Colloidal gold particles were prepared by adding 2.5 μg of antibody for each to 1 OD₅₂₀ of 40 nm colloidal gold (British Biocell International). After a 10 minute incubation, the gold was stabilized by the addition of bovine serum albumin and excess non-bound antibody removed by washing by centrifugation.

100 μL of dilutions of each extract were placed in wells of 48 well plates. 20 μL of colloidal gold at 2.0 OD₅₂₀ was added to each well, quickly mixed and one of the anti-PAT nitrocellulose strips added to each well. Solutions were allowed to wick up the strips for 10 minutes at which time the strips were removed and scored for color intensity relative to gradations of red on a color card.

### EXAMPLE 7

### Direct Bind Titration of Monoclonal Antibodies

An experiment was performed for the direct bind titration of monoclonal antibodies to PAT.

1. Two plates were coated with PAT antigen at 1.0 μg/mL on 0.1 M Carb pH 9.6 for one hour. Dump contents.

2. Block one hour with 200 μL PCT (PBS, 1% casein, pH 7.5), wash two by three times with PT (PBS, 0.05% Tween 20, pH 7.5).

3. Titrate monoclonal antibodies on plates with each coating antigen. Incubate one hour at 37° C. Titer in PCT. Wash as above.

4. Add 1:3000 dilution antibody in PCT to monoclonal antibody plates. Incubate 1 hour at 37° C. or over night at 4° C. Wash two by three times with PT.

5. Add 100 μL/well tetramethylbenzidine; incubate until sufficient color, read at OD₆₅₀.

All references cited herein are hereby incorporated by reference.

Modifications and variations of the present reagents, method and kit for detecting PAT protein will be obvious to those skilled in the art from the foregoing detailed description. Such modifications and variations are intended to come within the scope of the appended claims.

What is claimed is:

1. An immunoassay method for detecting phosphinothricin-N-acetyltransferase (PAT) protein in a sample comprising combining an antibody with the sample and detecting the formation of an antibody-PAT complex, wherein the antibody is immunoreactive with antigenic PAT peptides from two or more Streptomyces strains.

2. The method of claim 1, wherein the Streptomyces strains are selected from the group consisting of S. hygroscopicus and S. viridochromogenes.

3. The method of claim 1, wherein the antigenic PAT peptides comprise pat or bar gene peptides.

4. The method of claim 1, wherein the antigenic PAT peptides are immunoreactive with a monoclonal antibody selected from the group consisting of 98AD8, 98AY4, and 98BA12, or combinations thereof.

5. The method of claim 1, wherein the antibody is a polyclonal or monoclonal antibody.

6. The method of claim 1, wherein the antibody is a monoclonal antibody selected from the group consisting of 98AD8, 98AY4, and 98BA12, or combinations thereof.

7. Antigenic peptides comprising phosphinothricin-N-acetyltransferase (PAT) protein surface peptides that share epitopes across two or more Streptomyces strains.

8. The peptides of claim 7, wherein the Streptomyces strains are selected from the group consisting of S. hygroscopicus and S. viridochromogenes.

9. The peptides of claim 7, wherein the antigenic PAT peptides comprise pat or bar gene peptides.

10. The peptides of claim 7, wherein the antigenic PAT peptides are immunoreactive with a monoclonal antibody selected from the group consisting of 98AD8, 98AY4, and 98BA12, or combinations thereof.

11. An antibody for the detection of PAT protein, wherein the antibody is crossreactive with antigenic PAT peptides from two or more Streptomyces strains.

12. The antibody of claim 11, wherein the Streptomyces strains are selected from the group consisting of S. hygroscopicus and S. viridochromogenes.

13. The antibody of claim 11, wherein the antigenic PAT peptides comprise pat or bar gene peptides.

14. The antibody of claim 11, wherein the antibody is a polyclonal or monoclonal antibody.

15. The antibody of claim 11, wherein the antibody is a monoclonal antibody selected from the group consisting of 98AD8, 98AY4, and 98BA12, or combinations thereof.

16. The antibody of claim 11, wherein the antigenic PAT peptides are crossreactive with a monoclonal antibody selected from the group consisting of 98AD8, 98AY4, and 98BA12, or combinations thereof.

17. An immunoassay kit for the detection of PAT protein in a sample, said kit comprising two or more antibodies, each being immunoreactive with antigenic PAT peptides from two or more Streptomyces strains.

18. The kit of claim 17, wherein the Streptomyces strains are selected from the group consisting of S. hygroscopicus and S. viridochromogenes.

19. The kit of claim 17, wherein the antigenic PAT peptides comprise pat or bar gene peptides.

20. The kit of claim 17, wherein the antigenic PAT peptides are immunoreactive with a monoclonal antibody selected from the group consisting of 98AD8, 98AY4, and 98BA12, or combinations thereof.

21. The kit of claim 17, wherein the antibody comprises a polyclonal or monoclonal antibody.

22. The kit of claim 17, wherein the antibody is a monoclonal antibody selected from the group consisting of 98AD8, 98AY4, and 98BA12, or combinations thereof.

23. The kit of claim 17, wherein the antibodies are immobilized on a chromatogenic test strip.
